(19) United States
(12) Patent Application Publication
(10) Pub. No.: US 2021/0069236 A1
(43) Pub. Date: Mar. 11, 2021

(54) PREBIOTIC COMPOSITION

(71) Applicants: INSTITUT POLYTECHNIQUE DE GRENOBLE, GRENOBLE CEDEX 1 (FR); CENTRE NATIONAL DE LA RECHERCHE SCIENTIFIQUE -CNRS-, PARIS (FR); UNIVERSITE GRENOBLE ALPES, SAINT MARTIN D'HERES (FR); CENTRE HOSPITALIER UNIVERSITAIRE GRENOBLE ALPES, LA TRONCHE (FR)

(72) Inventors: Christine CHIRAT, GRENOBLE (FR); Bertrand TOUSSAINT, SAINT EGREVE (FR); Vivien DELOULE, GRENOBLE (FR); Jadwiga CHROBOCZEK, GRENOBLE (FR); Claire BOISSET-HELBERT, SAINT MARTIN D'URIAGE (FR)

(21) Appl. No.: 16/981,355

(22) PCT Filed: Mar. 25, 2019
(86) PCT No.: PCT/FR2019/050671

§ 371 Date: Sep. 16, 2020

(30) Foreign Application Priority Data
Mar. 23, 2018 (FR) 18 52534

(51) Int. Cl. A61K 31/736; A61P 1/14

(52) U.S. Cl. CPC A61K 31/736; A61P 1/14

(57) ABSTRACT

The field of the invention is that of prebiotic compositions.

In particular, the invention relates to a prebiotic composition comprising galactoglucomannans, and also the production method thereof.

The invention also targets the use of this prebiotic composition for increasing production of short-chain fatty acids by bacteria of the intestinal microbiota.

### FIELD OF THE INVENTION

The field of the invention is that of prebiotic compositions.

In particular, the invention relates to a prebiotic composition comprising galactoglucomannans, and also the production method thereof.

The invention also targets the use of this prebiotic composition for increasing production of short-chain fatty acids by bacteria of the intestinal microbiota.

### TECHNOLOGICAL BACKGROUND

Prebiotics are nutrients which promote the growth or activity of microorganisms of the intestinal microbiota. In particular, they support the growth of beneficial bacteria present in the gastrointestinal microbiota.

In Europe, the most commonly used prebiotics are fructo-oligosaccharides (FOS) produced from chicory root. Production of these prebiotics requires the use of a food resource and amble lands. Current production is therefore limited and directly competes with production of food resources. Additionally, the surface area of arable land is constantly decreasing. It would thus be attractive to propose prebiotics which come from nonfood resources in order to not compete with production of foodstuffs and to preserve arable land.

### Objectives

In these circumstances, the present invention aims to satisfy at least one of the objectives stated below.

One of the essential objectives of the present invention is to provide a prebiotic composition coming from nonfood resources.

One of the essential objectives of the present invention is to provide a prebiotic composition coming from available and abundant resources.

Another essential objective of the present invention is to provide an alternative prebiotic composition.

One of the essential objectives of the present invention is to provide an economical prebiotic.

Another essential objective of the present invention is to provide a prebiotic composition that is effective in terms of promotion of the growth of the microbiota.

Another essential objective of the present invention is to provide a prebiotic composition that is effective in terms of induction of the selective synthesis of short-chain fatty acids by the microbiota.

One of the essential objectives of the present invention is to provide a prebiotic composition with preventive and/or curative target.

Another essential object of the present invention is to provide a method for synthesis of a prebiotic composition which can be easily and economically implemented.

### BRIEF SUMMARY OF THE INVENTION

All or part of these objectives are reached by the present invention which relates to a prebiotic composition comprising galactoglucomannans having a degree of polymerization between 1 and 50, and having an acetyl level greater than or equal to 0.1% by mass relative to the total mass of the composition. This composition can be produced from lignocellulosic matter, in particular, from wood hemicelluloses.

The papermaking industry uses wood for extracting cellulose fibers and thus producing paper pulp. According to a standard process, cellulose is extracted from wood by high temperature alkaline processing (kraft process). The hemicelluloses and lignin breakdown products and other subproducts resulting from this processing are gathered in an effluent called “black liquor.”

It is to the inventors' credit that they discovered that the hemicelluloses extracted from wood by an autohydrolysis step applied to the wood before the kraft process can be used for producing a prebiotic composition.

Thus, it is possible to make use of the lignocellulosic matter, and therefore a non-food resource, for producing an effective prebiotic composition.

The invention also relates to a method for producing a prebiotic composition, which comprises the following steps:
a) autohydrolysis of lignocellulosic matter by thermal treatment in presence of water or steam;
b) purification of the hydrolysate of lignocellulosic matter obtained in step a).

The process is effective and economical because it can be incorporated in the papermaking process and in particular in the cellulose production factories present in many countries. Further, with this process a resource can be recovered which until now was rarely recovered and of low recovery value.

Another aspect of the invention is the use of a prebiotic composition for increasing the short-chain fatty acid production by bacteria of the intestinal microbiota.

Finally, the invention also relates to a prebiotic composition for therapeutic use, in particular for preventing or treating inflammatory diseases of the liver (non-alcoholic hepatic steatosis, hepatic fibrosis), chronic inflammatory diseases of the intestine (Crohn's disease, inflammatory colitis), systemic chronic inflammatory states, and metabolic imbalances (insulin resistance, dyslipidemia).

### Definitions

“Prebiotic composition” is understood, for example, to mean a composition which has a beneficial effect on the growth or activity of microorganisms of the intestinal microbiota.

“Galactoglucomannans” is understood, for example, to mean oligosaccharides comprising mannose, glucose and galactose units. These oligosaccharides generally comprise a main chain of mannoses linked by β-(1-4) glycosidic bonds with randomly interspersed glucose units, and, occasionally, galactoses linked by α-(1-6) glycosidic bonds in lateral chains. The glucose/mannose/galactose ratio varies according to the species in the following proportions 1/1.5 to 4.5/0 to 1. The hydroxyl groups in position C₂ and C₃ can be partially substituted by acetyl groups. A nonlimiting example of the structure of a galactoglucomannan chain is provided below (Fengel, D and Wegener, G. (1983) Wood: chemistry, ultrastructure, reactions. Walter de Gruyter, NY).

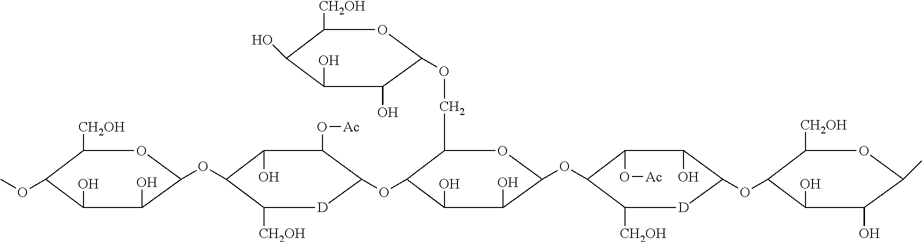

“Short-chain fatty acids” is understood to mean, for example, fatty acids having a carbon chain from 1 to 6 carbon atoms included. The following can be cited as examples of short-chain fatty acids: formic acid, acetic acid, propionic acid, butyric acid, isobutyric acid, valeric acid and isovaleric acid.

“Lignocellulosic matter” is understood to mean, for example, the principal constituent of the cell wall of plants principally composed of cellulose, hemicellulose, lignin and extractables such as polyphenol and terpenoids.

The “degree of polymerization” (DP) defines the length of a polymer chain. In the present disclosure, these terms designate the number of monomer units of saccharides making up an oligosaccharide or polysaccharide chain.

### DETAILED DESCRIPTION OF THE INVENTION

### Prebiotic Composition

The present invention relates in the first place to a prebiotic composition comprising galactoglucomannans having a degree of polymerization between 1 and 50, preferably between 1 and 35, and having an acetyl level greater than or equal to 0.1% by mass relative to the total mass of the composition, preferably greater than or equal to 4% and even more preferably greater than or equal to 6%.

According to an embodiment, the degree of polymerization of the galactoglucomannans is between 1 and 20 or between 1 and 15.

The acetyl level of the composition is expressed by mass relative to the total mass of the prebiotic composition. This acetyl level can be determined by calculating the acetic acid concentration difference before and after acid hydrolysis of the prebiotic composition. This hydrolysis can for example be done with a 3% solution of sulfuric acid (H₂SO₄) for 1 hour at 120° C. The acetyl level can be between 0.1 and 50%, between 2 and 25%, between 4 and 15%, or even between 6 and 10%. The acetyls principally come from acetylated galactoglucomannans.

The prebiotic composition comes from lignocellulosic matter, preferably from wood. Lignocellulosic matter can be chosen among resinous wood, deciduous would, recycled wood, recycled paper and cardboard, and mixtures thereof.

According to an embodiment, the prebiotic composition comes from one or more types of wood, preferably from one or more resinous types of wood.

According to an embodiment, the prebiotic composition comes from hemicellulose contained in lignocellulosic matter. Preferably, the prebiotic composition comes from hemicelluloses contained in wood.

The prebiotic composition may comprise at least 20% by mass of galactoglucomannans relative to the total mass of the composition. According to an embodiment, the prebiotic composition comprises at least 30% by mass, at least 40%, at least 50%, at least 60%, at least 70%, or at least 75% galactoglucomannans. According to an embodiment, the prebiotic composition comprises between 20 and 100% by mass of galactoglucomannans, between 50 and 99%, or between 75 and 98%.

According to a specific embodiment of the invention, the prebiotic composition also comprises lignin. The composition may comprise up to 20% by mass of lignin relative to the total mass of the composition. According to an embodiment, the composition comprises between 0.1 and 20% by mass of lignin, or between 0.2 and 5%.

According to an embodiment, the composition also comprises xylanes, for example between 0.1 and 35% of xylenes by mass relative to the total mass of the composition.

According to a preferred embodiment, the prebiotic composition coming from lignocellulosic matter comprises galactoglucomannans having a degree of polymerization between 1 and 35 and an acetyl level between 4 and 15%, preferably between 6 and 10%.

The composition may also comprise other compounds such as aromatic compounds.

The prebiotic composition has a marked promoter effect on the growth and/or activity of microorganisms from the intestinal microbiota. In particular, this composition stimulates the growth of beneficial bacteria from the intestinal microbiota. Further, the most acetylated galactoglucomannans are consumed later by the bacteria than the non-acetylated galactoglucomannans. In that way, a composition in which the galactoglucomannans have a high acetyl level serves to improve the growth of bacteria over a longer time than in the case of less acetylated or non-acetylated galactoglucomannans.

This composition has another advantageous effect, because it promotes the production of short-chain fatty acids in bacteria of the intestinal microbiota. In particular, this composition promotes the production of acetic acid, propionic acid, and butyric acid by the bacteria of the intestinal microbiota.

### Process for Producing a Prebiotic Composition

The invention also relates to a process for producing a prebiotic composition which comprises the following steps:
a) autohydrolysis of lignocellulosic materials by thermal treatment in presence of water or steam;
b) purification of the hydrolysate of lignocellulosic materials obtained in step a).

The step a) of autohydrolysis may be done at a temperature between 100 and 230° C., preferably between 150 and 80° C. and for a time between 20 minutes and 10 hours, preferably between 30 minutes and 2 hours. According to a preferred embodiment, the step a) is a step of autohydrolysis of one or more types of wood, preferably one or more resinous types of wood. In this case, after this step a) a hydrolysate of lignocellulosic matter results which comprises hemicellulose from wood, including galactoglucomannans.

According to a preferred embodiment, the step a) is done in a closed, pressurized reactor. Pressure in the reactor corresponds to the saturating vapor pressure of water, which varies with the chosen temperature. According to an embodiment, the lignocellulosic matter is in the form of shavings.

Step b) of purification of the hydrolysate of lignocellulosic matter may comprise several phases.

According to a preferred embodiment, the step b) comprises a phase of processing with activated charcoal and/or a phase of nano- or ultra-filtration and/or a phase of precipitation in a solvent.

Step b) may also comprise a step of centrifuging and/or microfiltration for removing insoluble particles from the hydrolysate.

According to an embodiment of the process, the step b) comprises a phase of processing with activated charcoal and a phase of nano- or ultra-filtration.

According to another embodiment of the process, the step b) comprises a phase of processing with activated charcoal and a phase of precipitation in a solvent.

According to another embodiment of the process, the step b) comprises a phase of nano- or ultra-filtration and a phase of precipitation in a solvent.

According to another embodiment of the process, the step b) comprises a phase of processing with activated charcoal, a phase of nano- or ultra-filtration and a phase of precipitation in a solvent.

The various purification phases can be done in any order.

The phase of nano- or ultra-filtration comprises the step of filtration of the hydrolysate over a membrane at the determined cutoff threshold.

According to an embodiment, the cutoff threshold of the membrane is included between 0.2 kDa and 30 kDa. For example, the cutoff threshold can be 0.2 kDa, 0.5 kDa, 1 kDa, 5 kDa, 8 kDa, 10 kDa, 20 kDa or 30 kDa. The person skilled in the art is able to choose the cutoff threshold according to the desired degree of polymerization of the galactoglucomannans.

It is also possible to combine several phases of nano- or ultrafiltration.

The phase of precipitation in a solvent can be done with an organic solvent, a mixture of organic solvents, or a mixture of organic solvent(s) and water.

According to an embodiment, the phase of precipitation in the solvent is done with acetone, ethanol, a mixture of acetone and methanol, or a mixture of ethanol and water.

According to a specific embodiment of the process, the process also comprises a step of enzymatic processing of the hydrolysate resulting from step a), or of the prebiotic composition resulting from step b). This processing can be done with one or several enzymes, for example with one or several enzymes from the family of mannanases, xylanases, acetylesterases, or glucuronidases.

This processing can be used for reducing the size of the oligosaccharides and/or polysaccharides. It can also be used to modulate the acetyl level of the galactoglucomannans, by acetylating free hydroxyls or by breaking acetyl groups already present.

For example, it is possible to reduce the acetyl level of the prebiotic composition by a step of partial enzymatic deacetylation of the galactoglucomannans.

The invention also relates to a prebiotic composition which could be obtained by the above process.

### Use of the Prebiotic Composition

The invention also relates to the use of the prebiotic composition according to the invention for increasing the production of short-chain fatty acids by bacteria of the intestinal microbiota.

According to an embodiment, the short-chain fatty acids are chosen among acetic acid, propionic acid, butyric acid and mixtures thereof.

An object of the invention is also a prebiotic composition according to the invention for the use in preventive and/or curative therapy. The prebiotic composition according to the invention has a beneficial effect on the growth and/or activity of microorganisms in the intestinal microbiota, so it can be used in the treatment and/or prevention of certain diseases.

An object of the invention is also a prebiotic composition according to the invention for use in the prevention and/or treatment of inflammatory diseases of the liver (non-alcoholic hepatic steatosis, hepatic fibrosis), chronic inflammatory diseases of the intestine (Crohn's disease, inflammatory colitis), systemic chronic inflammatory states, or metabolic imbalances (insulin resistance, dyslipidemia).

### BRIEF DESCRIPTION OF THE DRAWINGS

FIG. 1 shows the degrees of polymerization of the various prebiotic compositions according to Example 1 and a control composition of Fructo-Oligosaccharides (FOS).

FIG. 2A represents the curves of ATP production from E. coli as a function of time according to Example 2. FIG. 2B represents the curves of ATP production from B. adolescentis as a function of time according to Example 2. FIG. 2C represents the curves of ATP production from A. muciniphila as a function of time according to Example 2. FIG. 2D represents the curves of ATP production from L. salivarius as a function of time according to Example 2.

FIG. 3 shows the distribution of the bacteria from the cecum of mice, according to their phylum, according to Example 3 (gp control=average of the control mice, gp hemicell=average of the mice fed with the prebiotic composition according to the invention).

FIG. 4 shows the quantity of short-chain fatty acids in the cecum of mice according to Example 3 (a=acetic acid, b=butyric acid, c=propionic acid, d=formic acid, gp control=control mice, gp hemicell=mice fed with the prebiotic composition).

### EXAMPLES

### Example 1: Preparation of Prebiotic Composition

a) Wood Hydrolysate

The wood used in this study is a mixture of resinous shavings (Scotch pine, black pine, Aleppo pine, Douglas fir and spruce).

Wood shavings and distilled water are put in a reactor with a water/wood ratio of 3 (300 mL of water for 100 g of kiln-dried wood). The mixture is next heated in the closed reactor 170° C. for one hour. After cooling to ambient temperature, an autohydrolysate of lignocellulosic matter (AutoH) results.

b) Purification of the Hydrolysate

Different purifications were tested:
processing with activated charcoal followed by precipitation with an acetone:methanol mixture (PP Acet)
processing with activated charcoal followed by precipitation with an ethanol:water mixture (PP eth)
ultrafiltration followed by treatment with activated charcoal (AutoH UC)

### PP Acet

The hydrolysate is treated with activated charcoal (10 g/L). This step is followed by two successive precipitations with an acetone:ethanol (9:1) mixture, and then a lyophilization of the prebiotic composition.

### PP Eth

The hydrolysate is treated with activated charcoal (10 g/L). This step is followed by two successive precipitations with an ethanol:water (9:1) mixture, and then a lyophilization of the prebiotic composition.

### AutoH UC

The hydrolysate is filtered on a 0.5 kDa cutoff threshold membrane (Merck Millipore, regenerated cellulose) by using Amicon® Stirred Cells (Merck Millipore). The hydrolysate is then treated with activated charcoal (40 g/L). Then the prebiotic composition is lyophilized.

c) Analysis of the Various Prebiotic Compositions

The monomers were quantified by High-Performance Anion-Exchange Chromatography with Pulsed Amperometric Detection (HPAE-PAD Dionex™ ICS-5000 with a CarboPac™ PA10 column; 25° C.). The oligomer concentration in the fractions and the initial autohydrolysate was calculated from the increase of the monosaccharide concentration after a post-hydrolysis acid (120° C./1 hour, 3% H₂SO₄).

### AutoH

57.6% oligomers/total dry mass of the hydrolysate with a 0.28:1 arabinoxylane:galactoglucomannans ratio and 28.7% of monomer relative to the total carbohydrates. The galactoglucomannans concentration is therefore 45% by mass relative to the total mass of the composition.

### PP Acet

91.6% oligomers/total dry mass of the prebiotic composition with a 0.18:1 arabinoxylane:galactoglucomannans ratio and 5.7% of monomer relative to the total carbohydrates. The galactoglucomannans concentration is therefore 77.6% by mass relative to the total mass of the composition.

### PP Eth

94.5% oligomers/total dry mass of the prebiotic composition with a 0.07:1 arabinoxylane:galactoglucomannans ratio and 0.6% of monomer relative to the total carbohydrates. The galactoglucomannans concentration is therefore 88.3% by mass relative to the total mass of the composition.

### AutoH UC

98.9% oligomers/total dry mass of the prebiotic composition with a 0.20:1 arabinoxylane:galactoglucomannans ratio and 0.6% of monomer relative to the total carbohydrates. The galactoglucomannans concentration is therefore 82.4% by mass relative to the total mass of the composition.

The breakdown products (furfural, hydroxymethyl furfural (HMF), formic acid and also acetic acid) were quantified by HPLC. The purified hydrolysates no longer contain furfural, HMF, formic acid or acetic acid.

The quantity of acetyl groups present on the solubilized hemicelluloses was determined by calculating the difference in the acetic acid concentration before and after a post-hydrolysis acid (120° C./1 hour, 3% H₂SO₄). For the unpurified autohydrolysate, the acetyl level is about 4%. For the purified prebiotic compositions PP acet and PP eth, the acetyl levels are respectively 8.3 and 8.9%.

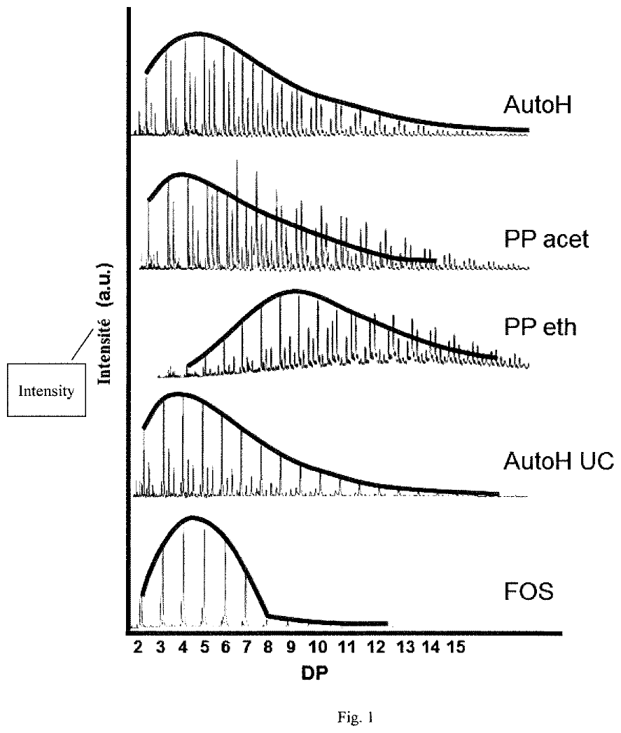

The degrees of polymerization of the oligosaccharides and polysaccharides were measured by mass spectroscopy (MS) with matrix-assisted laser desorption ionization-time-of-flight (MALDI-TOF) technology and GPC MALS (gel permeation chromatography-multi-angle laser scattering). FIG. 1 shows the degrees of polymerization of the oligosaccharides and polysaccharides of the various prebiotic compositions.

### Example 2: Test of the Various Prebiotic Compositions on Growth of the Bacteria

Adherent-invasive Escherichia coli (AJEC) LF82 and Bifidobacterium adolescentis CFPL 15,196, were isolated from human feces in the Lille (France) pharmacy department. Akkermansia muciniphila (ATCC® BAA-835™) was purchased from ATCC. Lactobacillus salivarius CIP 103,140 was purchased from the Institut Pasteur Collection. All cultures were done under anaerobic atmosphere made up of 10% H₂, 5% CO₂ and 85% N₂ (Anaerogaz, Linde), in a medium containing deoxygenated soy broth without dextrose (TSWD, Tryptic soy without dextrose, Becton-Dickinson). TSWD was supplemented with 1% (WN) of the various prebiotic compositions according to Example 1. The PP eth composition was also tested at 3% (WN). The negative control contained only TSWD. The positive controls were made up of a prebiotic fructo-oligosaccharide at a 1% concentration (FOS P95, BENEO-Orafti, Belgium) with an average degree of polymerization of 4 (from 2 to 9) or of 1% glucose added to TSWD.

a) Measurement of the Quantity of ATP

The production of intracellular adenosine triphosphate (ATP) was measured.

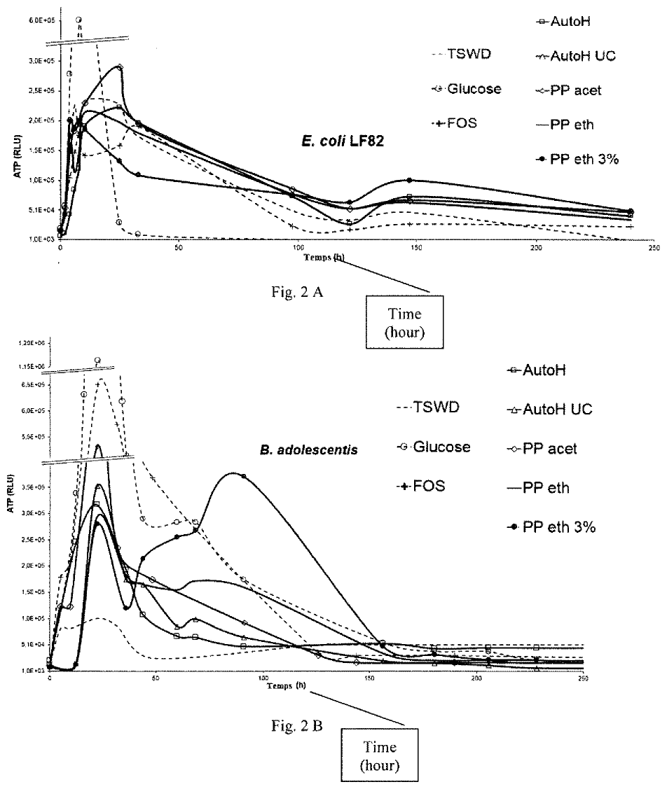

FIGS. 2A to 2D show the quantity of ATP produced by the various bacteria. These results show that the compositions according to the invention really are prebiotic compositions because they support the growth of beneficial bacteria from the gastrointestinal microbiota, like B. adolescentis, A. muciniphila and L. salivarius. Further, the growth of harmful bacteria from the gastrointestinal microbiota, like E. coli LF82 is not supported.

b) Measurement of the Quantity of Short-Chain Fatty Acids

Short-chain fatty acids are extracted from supernatant bacterial samples by a liquid-liquid extraction and then analyzed by HPLC-UV.

The results show that the prebiotic compositions according to the invention promote the production of short-chain fatty acids, including formic acid, acetic acid and propionic acid.

c) Influence of the Degree of Acetylation

The medium in which the bacteria are cultured is analyzed by mass spectroscopy (MALDI TOF) at various reaction times. The results of these analyses show that the non-acetylated galactoglucomannans are consumed first by the bacteria from the beginning of the culture. The most acetylated galactoglucomannans are consumed later by the bacteria. This explains why the growth effect on the bacteria is obtained longer than for the FOS control.

### Example 3: In Vivo Test on Mice of a Prebiotic Composition

Tube feeding of mice with an ethanol precipitated fraction (PP eth) according to example 1 and analysis of 16S rRNA.

This experiment was evaluated and authorized by the Grenoble ethics committee and the French government (APAFIS number 8502-2016122009.36117). The animals were housed at the Plateforme de Haute Technologie Animale, Université Grenoble Alpes, under authorization number: C3851610006. Female mice C57BL/6N (five weeks old) were purchased from Janvier SA (Le Genest-Saint-Isle, France) and were housed in groups of four mice per cage with unrestricted access to wood and water. The mice were fed for one week with the control diet of A04 (SAFE, Villemoisson-sur-Orge, France). Next, eight control mice were fed for three weeks with A04 whereas eight other mice were fed with A04 with prebiotic composition (PP eth) added at a rate of 0.3 g/day per mouse, dissolved in water. To do that, the lyophilized fraction precipitated from ethanol was dissolved in water to reach 86 g/L and filtered through a 0.2 μm membrane. The feed bottles (100 mL) were changed every 2 to 3 days and replaced with a freshly prepared solution. At the end of the experimental period, the mice were euthanized with isoflurane. A median ventral incision was made for excising the cecum and the colon. The cecal content was collected and immediately frozen in liquid nitrogen. Portions of cecal content were prepared with 200 μL of PBS for a cecal level of 40 mg, by homogenization. The fecal slurry was centrifuged at 35,000 g for 20 minutes at 4° C. and the supernatant was collected and sterilized by filtration (0.22 m).

a) Analysis of the 16S rRNA

The analysis of the 16S rRNA (and extraction of the RNA) was subcontracted to Vaiomer (Vaiomer SA, Labdge, France). The data resulting from this analysis were not only bacterial abundance data, but also the alpha diversity, meaning the richness (the number of unique bacterial taxons in the samples).

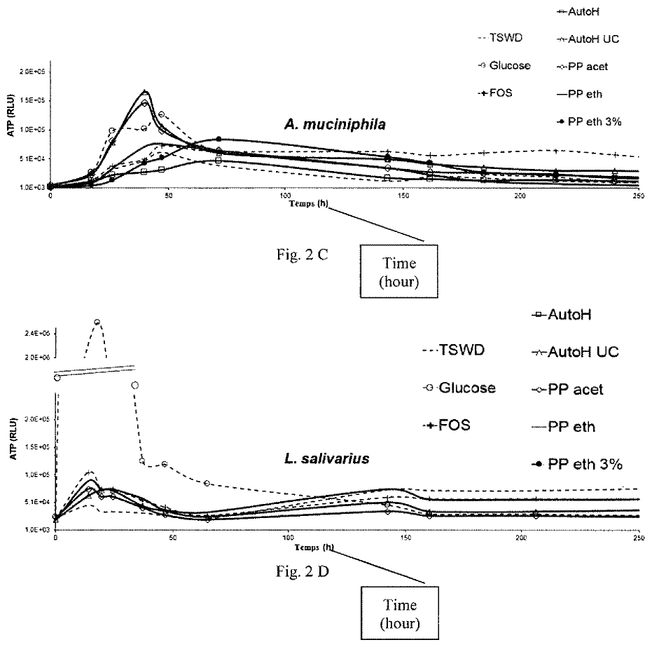

FIG. 3 shows the distribution of the various bacterial classes in the cecum. These results show that the mice who received the prebiotic composition according to the invention had a different distribution of bacterial classes compared to the mice who did not receive the prebiotic composition.

In particular, the mice who received the prebiotic composition according to the invention had a larger proportion of beneficial bacteria present in the gastrointestinal microbiota and a lower proportion of harmful bacteria present in the gastrointestinal microbiota.

b) Measurement of the Quantity of Short-Chain Fatty Acids

The short-chain fatty acids were analyzed in the cecum content according to the protocol from Example 2b.

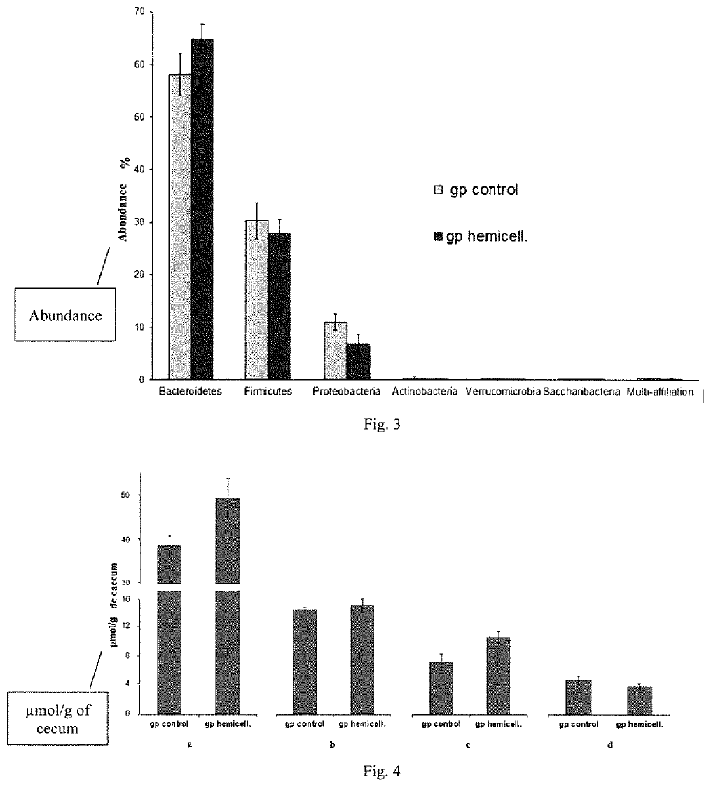

FIG. 4 shows the quantity of short-chain fatty acids contained in the cecum. These diagrams show that the mice who received the prebiotic composition according to the invention produced more short-chain fatty acids, in particular, more acetic acid and more propionic acid, compared to the mice who did not receive the prebiotic composition.

### Example 4. Effect of Enzymatic Hydrolysis on the Size of the Oligosaccharides

The wood hydrolysate prepared such as presented in example 1 (AutoH) was subject to enzymatic hydrolysis by using a mannanase (35 mg enzymes/gram of oligosaccharides) and a cellulase (500 and 3,000 EGU/g of oligosaccharides). The average molecular mass (Mw) measured by GPC MALS showed the following results:

Mw of the oligosaccharides in the starting hydrolysate: 2.3 kDa
Mw of oligosaccharides after treatment by a mannanase: 1.7 kDa
Mw of oligosaccharides after treatment by a cellulase respectively at 500 and 3,000 EGU/g: 1.7 and 1.0 kDa respectively.

These results show that it is possible to reduce the size of the oligosaccharides contained in the hydrolysate by enzymatic treatment.

1. A prebiotic composition comprising galactoglucomannans having a degree of polymerization between 1 and 50, preferably between 1 and 35, and in that it has an acetyl level greater than or equal to 0.1% by mass relative to the total mass of the composition, preferably greater than or equal to 4%, and even more preferably greater than or equal to 6%.

2. The prebiotic composition according to claim 1, said composition coming from lignocellulosic matter.

3. The prebiotic composition according to claim 1, said composition coming from one or more types of wood, preferably from one or more resinous types of wood.

4. The prebiotic composition according to claim 1, wherein the galactoglucomannans concentration is greater than or equal to 20% by mass relative to the total mass of the composition.

5. The prebiotic composition according to claim 1, said composition further comprising lignin.

6. The prebiotic composition according to claim 1, said composition promoting the production of short-chain fatty acids in bacteria of the intestinal microbiota.

7. A process for producing a prebiotic composition according to claim 1, comprising the following steps:
a) autohydrolysis of lignocellulosic materials by thermal treatment in presence of water or steam;
b) purification of the hydrolysate of lignocellulosic materials obtained in step a).

8. The process according to claim 7, wherein the step a) of autohydrolysis is done at a temperature between 100 and 230° C., preferably between 150 and 180° C. and for a time between 20 minutes and 10 hours, preferably between 30 minutes and 2 hours.

9. The process according to claim 7, wherein the step b) comprises a phase of processing with activated charcoal and/or a phase of nano- or ultra-filtration and/or a phase of precipitation in a solvent.

10. The process according to claim 7, wherein the step a) is a step of autohydrolysis of one or more types of wood, preferably one or more resinous types of wood.

11. A method for increasing the production of short-chain fatty acids by bacteria of the intestinal microbiota, using a prebiotic composition according to claim 1.

12. (canceled)

13. A method for preventing and/or treating inflammatory diseases of the liver, chronic inflammatory diseases of the intestine, systemic chronic inflammatory states, or metabolic imbalances, using a prebiotic composition according to claim 1.
